(19) United States
(12) Patent Application Publication
(10) Pub. No.: US 2025/0312556 A1
(43) Pub. Date: Oct. 9, 2025

(54) CATHETER ASSEMBLY FOR REGIONAL ANAESTHESIA

(71) Applicant: B. Braun Melsungen AG, Melsungen (DE)

(72) Inventors: Cristiane Henschel, Kassel (BR); Jacqueline Berg, Melsungen (DE); Johannes Bolz, Kassel (DE); Benjamin Claus, Kassel (DE); Manuel Freudenstein, Lohfelden (DE); Christian Hohmann, Edermuende (DE); Roman Klotzbach, Baunatal (DE)

(21) Appl. No.: 19/098,019

(22) Filed: Apr. 2, 2025

(30) Foreign Application Priority Data
Apr. 3, 2024 (DE) 10 2024 109 245.9

(51) Int. Cl. A61M 19/00; A61M 25/00; A61M 39/10

(52) U.S. Cl. CPC A61M 19/00; A61M 25/0082; A61M 39/10; A61M 2202/048; A61M 2205/3334

(57) ABSTRACT

A catheter assembly for regional anaesthesia includes a catheter assembly with a capillary housing, a capillary tube, and a catheter tube. The catheter tube is inserted proximally in the capillary tube and protrudes distally out of the capillary tube. The catheter tube is arranged in the capillary tube with limited linear displaceability. The catheter assembly also includes a manually actuatable clamping device for detachable fixing of the catheter tube relative to the capillary tube and the capillary housing. The catheter assembly further includes an adjustment device on the capillary housing that ensures a graduated or continuous displacement of the catheter tube in the capillary tube. Moreover, the catheter assembly includes at least one manually grippable radial projection on the catheter tube that is displaceably guided along the capillary housing.

### CROSS-REFERENCE TO RELATED APPLICATION

This application claims priority under 35 U.S.C. § 119 to German Application No. 10 2024 109 245.9, filed on Apr. 3, 2024, the content of which is incorporated by reference herein in its entirety.

### FIELD

The present disclosure relates to a catheter assembly for regional anaesthesia, comprising a capillary housing, on which distally a capillary tube is provided, and comprising a catheter tube, which is inserted proximally in the capillary tube and protrudes distally partially out of the capillary tube, wherein the catheter tube is arranged in the capillary tube with limited linear displacement, and comprising a manually actuatable clamping device for detachable fixing of the catheter tube relative to the capillary tube and the capillary housing.

### BACKGROUND

Such a catheter assembly is known from DE 10 2022 205 130 A1. The known catheter assembly has a capillary housing and a capillary tube fixedly attached to the capillary housing. A catheter tube in the form of a hose is inserted in the capillary tube and protrudes axially beyond the capillary tube at a distal tube end of the capillary tube. To fix the catheter tube relative to the capillary housing, a fixing apparatus having an actuating element and a clamping element is provided. The clamping element is mounted on the capillary housing. The actuating element is movably mounted relative to the capillary housing and operatively connected to the clamping element with transmission of force and/or movement, in order to make it possible to axially clamp the catheter tube, which is in the form of a hose, relative to the capillary tube or enable an axial movability of the catheter tube, which is in the form of a hose, relative to the capillary tube.

### SUMMARY

An object of the present disclosure is to provide a catheter assembly which is of the type mentioned in the introduction and can be operated easily.

This object is achieved in that an adjustment device is additionally provided on the capillary housing and ensures a graduated or continuous displacement of the catheter tube in the capillary tube, wherein at least one manually grippable radial projection, in particular an actuator, which is guided with limited displaceability along the capillary housing, is provided on the catheter tube. The solution according to the present disclosure is intended for regional anaesthesia. The adjustment device provided on the capillary housing makes it possible to adjust an advancement of a catheter tube portion projecting distally out of the capillary tube and then fix the adjusted advancement position by means of the clamping device. Both the adjustment device and the clamping device can be operated with one hand by a user, and therefore the user's other hand is free to perform an additional function, such as in particular holding and guiding an ultrasound instrument. By modifying the advancement, a tip of the catheter tube can be guided up particularly close to the location to be anaesthetized, in particular to nerves or nerve plexuses, in order to obtain a high effectiveness of the regional anaesthesia. The catheter tube may be at least partially in the form of a hose. Moreover, an extension hose can be proximally joined to the catheter tube. If the extension hose is pushed externally onto a proximal end of the catheter tube, the distal face end of the extension hose forms the radial projection according to the present disclosure. As an alternative, a bead or a similar actuator can be fastened on the catheter tube, in order to serve as a radial projection. The term “radial(ly)” is with respect to a longitudinal axis of the catheter tube. The term “catheter tube” also includes such an extension hose connected proximally to the catheter tube guided in the capillary tube, and therefore the projection, in particular the actuator, can also be provided on the extension hose. The actuator preferably encloses a region of the catheter tube in particular at the level of the capillary housing in force-fitting, form-fitting or integrally bonded fashion, such that an adjustment of the actuator necessarily brings about a complementary displacement of the catheter tube. If the catheter tube is displaced in graduated fashion, at least two stepped profiles in which the actuator can detachably latch are preferably provided on the capillary housing. If the catheter tube can be displaced continuously, any desired positionings within a limited advancement portion, relative to the capillary tube, of preferably between 10 and 30 mm are possible. The projection can be manually gripped, such that the catheter tube can be displaced with a finger of one hand.

In one configuration of the present disclosure, it is provided that a manually operable actuating element is operatively connected to the projection, in particular the actuator, and is movably arranged on the capillary housing for a displacement of the projection, in particular the actuator, along the capillary housing. The outer contour or a surface shape of the projection, in particular of the actuator, can additionally also form the actuating element, or the actuating element is a separate functional part operatively connected to the projection, in particular the actuator, or is a separate functional portion fixedly connected to, in particular formed in one piece with, the projection and/or the actuator.

In a further configuration of the present disclosure, the actuating element is fixedly connected to the actuator. The actuating element may be integrally formed on the actuator or fastened to the actuator in another way, in particular by latching. Advantageously, the actuating element can be formed by an actuator contour or surface adapted for being manually gripped or touched by at least one finger of a user's hand, with the result that the actuator and the actuating element are made of one and the same material and are one and the same component. The surface can have different haptics to other faces of the actuator, in order to determine the function of the actuating element. As an alternative, the actuating element can be an independent functional part fixedly connected to the actuator in one piece or via additional fastening members.

In a further configuration of the present disclosure, the actuating element indirectly interacts with the actuator with transmission of movement. In this configuration, there is no fixed connection between the actuating element and the actuator. Instead, the actuating element is movably mounted on the capillary housing and coupled to the actuator by means of a drive transmission member, such as in particular a gear mechanism.

In a further configuration of the present disclosure, the actuating element is displaceably mounted on the capillary housing. To this end, a linear guide track, in particular at least one guide bar or at least one guide groove, is provided on the capillary housing, and the actuating element has at least one complementary guide profile which interacts with the linear guide track, in order to obtain the displaceability of the actuating element relative to the capillary housing. The linear guide thus formed is limited, preferably to an adjustment travel between 10 and 30 mm.

In a further configuration of the present disclosure, the actuating element is manually detachably retained on the capillary housing in force-fitting or form-fitting fashion in at least two different functional positions. This configuration enables graduated displaceability of the actuating element and hence also of the actuator and of the catheter tube relative to the capillary tube. With preference, the actuating element and the capillary housing have mutually complementary latching profiles which interact in form-fitting or force-fitting fashion, in order to define a corresponding advancement position for the catheter tube. The force-fitting or form-fitting functional positions can be overridden manually by a user's hand, such that the user, after detaching the clamping device, can easily displace the actuating element with the same hand into the respective next functional position.

In a further configuration of the present disclosure, mutually complementary latching profiles are provided on the actuating element and on the capillary housing, in order to manually detachably secure the at least two different functional positions. The latching profiles may be formed by latching recesses on the capillary housing and by a complementary configuration of latching cams or latching lugs on an outer contour of the actuating element and/or the actuator, with the result that the actuating element and/or the actuator can form-fittingly latch into a respective latching recess on the capillary housing.

In a further configuration of the present disclosure, a linear guide for displacement of the actuating element is provided on the capillary housing, and the actuating element is in the form of an actuating slide which is displaceable along the linear guide. The actuating slide can be one and the same component with the actuator. This configuration implements a continuous displaceability of the catheter tube relative to the capillary housing and relative to the capillary tube in the limited region of advancement.

In a further configuration of the present disclosure, the actuating element is rotatably mounted on the capillary housing, in order to be able to displace the actuator and thus the catheter tube along the capillary housing and in the capillary tube. This results in an indirect displacement of the catheter tube by means of a manual rotation of the actuating element.

In a further configuration of the present disclosure, the actuating element is rotatably mounted on the capillary housing coaxially with a longitudinal axis of the capillary tube and is coupled to the actuator by means of a screw drive. This is a space-saving configuration which is easy to operate, since a desired advancement of the catheter tube is achieved by a rotation of the actuating element, this rotation being coaxial with a longitudinal axis of the catheter tube.

In a further configuration of the present disclosure, the clamping device has a clamping member, which force-fittingly acts on the catheter tube, and an operating member, which is movably mounted on the capillary housing, elastically deforms the clamping member in a clamping position, in order to force-fittingly clamp the catheter tube, and in a release position releases the clamping member, with the result that the clamping member returns to its unloaded initial position, in which the catheter tube is movable relative to the clamping member. The clamping member can coaxially enclose the catheter tube. As an alternative, the clamping member can act on the catheter tube radially from one side. In all variants, the operating member acts on the clamping member in such a way that the clamping member radially elastically clamps the catheter tube relative to a longitudinal axis of the catheter tube.

In a further configuration of the present disclosure, the operating member is pivotably or rotatably mounted on the capillary housing. The operating member may be in the form of a lever, a rotary bayonet member, a pivotable sleeve portion, or the like.

### BRIEF DESCRIPTION OF THE DRAWINGS

Further advantages and features of the present disclosure will become clear from the following description of preferred exemplary embodiments of the present disclosure, which are shown in the drawings.

FIG. 1 schematically shows a perspective illustration of a first embodiment of a catheter assembly according to the present disclosure;

FIGS. 2a and 2b show a further embodiment of a catheter assembly according to the present disclosure in two different functional positions;

FIGS. 3a and 3b show a schematic perspective illustration of two different functional positions of a further embodiment of a catheter assembly according to the present disclosure;

FIG. 4 shows a schematic illustration, in longitudinal section, of a further embodiment of a catheter assembly according to the present disclosure;

FIG. 5 shows a perspective schematic illustration of a further embodiment of a catheter assembly according to the present disclosure;

FIGS. 6 and 7 show perspective diagrams of two different actuators, which are fastened on a catheter tube of a respective embodiment of a catheter assembly according to the present disclosure;

FIG. 8 shows a schematic, perspective illustration of a further embodiment of a catheter assembly according to the present disclosure;

FIG. 9 shows a schematic illustration, in longitudinal section, of a partial region of a further embodiment of a catheter assembly according to the present disclosure;

FIG. 10 shows a perspective, schematic, partially cut-away illustration of a partial region of a further embodiment of a catheter assembly according to the present disclosure; and

FIGS. 11 to 13 schematically show further embodiments of a catheter assembly according to the present disclosure.

### DETAILED DESCRIPTION

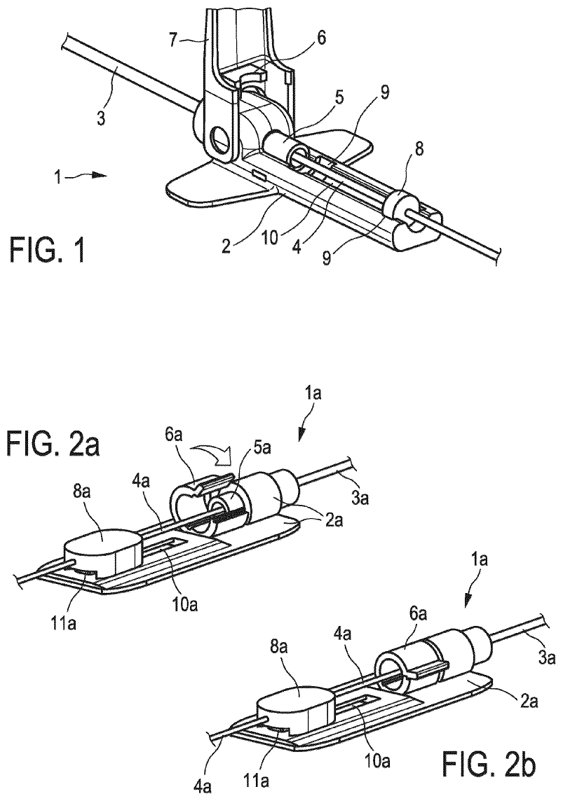

A catheter assembly 1 according to FIG. 1 is intended for regional anaesthesia in the field of medical technology. The catheter assembly 1 comprises a capillary housing 2, which is made of plastic. A capillary tube 3 is fastened to a distal end of the capillary housing 2, also referred to as capillary attachment, and is provided distally with a tip, this not being illustrated in more detail, which is subcutaneously introduced into a patient in the ready-for-use state. In order to be able, for regional anaesthesia, to come relatively close to individual nerves that are to be inhibited, the catheter assembly 1 additionally comprises a catheter tube 4, which is pushed into the capillary tube 3 proximally in the distal direction and a distal tip and also an adjoining advancement portion of which protrude distally out of the capillary tube 3. A length of the advancement portion, protruding beyond the distal tip of the capillary tube 3, of the catheter tube 4 can be adjusted in a way described in more detail below. The catheter tube 4 extends in the longitudinal direction through the capillary housing 2 and can be connected at its proximal end, which is not illustrated, to an extension hose. The catheter tube 4 is made of plastic in the form of a flexible hose.

The advancement length, protruding distally out of the capillary tube 3, of the catheter tube 4 can be adjusted by means of an adjustment device, described in more detail below. In the case of the catheter assembly 1 according to FIG. 1, for this purpose an actuator 8 in the form of a sleeve in the form of a bead is fastened on an outer surface of the catheter tube 4. The actuator 8 encloses the catheter tube 4 coaxially and has an at least largely cylindrical outer surface. The capillary housing 2 has two semi-cylindrical depressions 9, which are spaced apart from one another in the longitudinal direction of the catheter tube 4. The recesses 9 are matched to the outer surface of the actuator 8, in order to allow the actuator 8 to be detachably latched in one of these recesses 9. The two mutually spaced-apart recesses 9 are interconnected via a channel-like linear guide 10, which-like the recesses 9—is an integral constituent part of the capillary housing 2. In the position illustrated in FIG. 1, the catheter tube 4 is positioned in the longitudinal direction relative to the capillary housing 2 by the actuator 8 dipping in the proximal recess 9 in form-fitting fashion. If an advancement of the catheter tube 4 relative to the capillary tube 3 is to be modified, the actuator 8 can be easily raised by a user by means of two fingers of one hand and displaced distally along the capillary housing 2 along the linear guide 10, which is in the form of a channel, until the actuator 8 dips into the second recess 9 of the capillary housing 2. The catheter assembly 1 accordingly constitutes a possible way of advancing the catheter tube 4 in two stages.

In order to be able to additionally fix the catheter tube 4 relative to the capillary housing 2, a clamping device is provided which has an elastically deformable clamping member 5 that coaxially encloses the catheter tube 4 in the manner of a sleeve and is embedded in the capillary housing 2 distally next to the distal recess 9. In a release position, illustrated in FIG. 1, of the clamping device, the clamping member 5 encloses the catheter tube 4 with a radial spacing. This constitutes the unloaded initial position of the clamping member 5. In order to clamp the catheter tube 4 by means of the clamping member 5, an operating member 7 is provided, which in FIG. 1 is in the form of an operating lever pivotably mounted on the capillary housing 2 and which is provided with a force transmission portion 6 that radially elastically deforms the clamping member 5 from above—with respect to the illustration in FIG. 1—as the operating member 7 pivots closed. This causes the clamping member 5 to be pressed radially against the outer surface of the catheter tube 4, as a result of which the desired clamping action that axially fixes the catheter tube 4 relative to the capillary housing 2 is obtained. It is also possible to clamp the catheter tube 4 relative to the capillary housing 2 by means of the clamping member 5 in an intermediate position of the actuator 8 in the form of a bead, in which intermediate position the actuator 8 rests in the linear guide 10, which is in the form of a channel, between the two recesses 9 of the capillary housing 2.

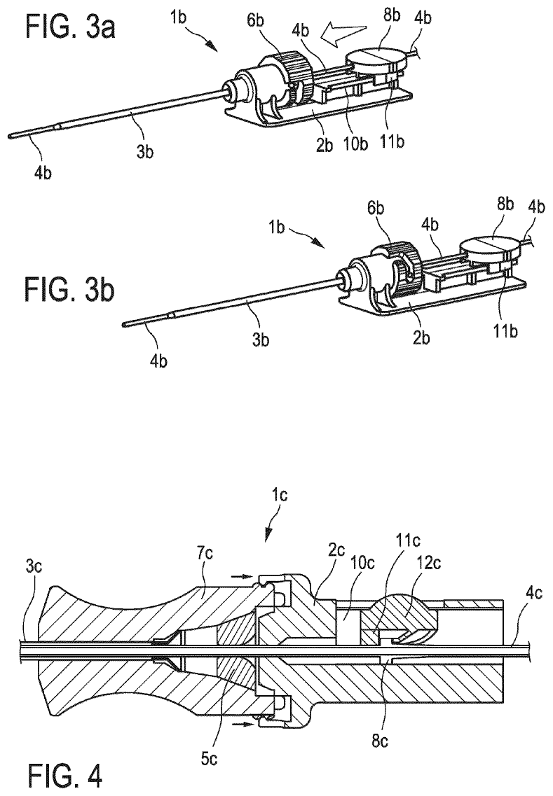

The catheter assembly 1a according to FIGS. 2a and 2b corresponds to the catheter assembly 1 according to FIG. 1 in terms of the functional structure. Components and portions which have the same function are therefore provided with the same reference signs with addition of the letter a. The catheter assembly 1a also analogously serves for regional anaesthesia. To avoid repetitions, reference is additionally made to the statements regarding the catheter assembly 1 according to FIG. 1. The same also applies for the embodiments of catheter assemblies according to the present disclosure that are described below by means of FIGS. 3a to 10. There as well, respective components or portions that have the same function are provided with the same reference signs with addition of a respective subsequent lower-case letter b to i. The use of these catheter assemblies 1b to 1g corresponds to the use of the catheter assemblies 1 and 1a, and therefore, to avoid repetitions, reference is additionally also made, with respect to these embodiments which are described in more detail below, to the above statements regarding the catheter assembly 1.

The catheter assembly 1a comprises a capillary housing 2a, which is distally fixedly connected to a capillary tube 3a. A catheter tube 4a protrudes coaxially in the distal direction through the capillary tube 3a. The catheter tube 4a is enclosed by an actuator 8a, which is mounted by means of a guide element 11a in a slot-like linear guide 10a with delimited displaceability along the capillary housing 2a. The actuator 8a is formed in the manner of a block and fixedly connected to the catheter tube 4a. The actuator 8a additionally forms an actuating element within the meaning of the present disclosure by way of its outer contour, which can be manually gripped by a user's hand. The linear guide 10a is provided in a support plate portion of the capillary housing 2a and limits a linear displaceability of the actuator 8a to an advancement travel which is preferably between 15 mm and 30 mm. The linear guide 10a extends in the longitudinal direction of the capillary housing 2a and hence parallel to the catheter tube 4a. The guide element 11a is held in the linear guide 10a of the capillary housing 2a in the manner of a slotted guide bar, wherein the guide is configured such that the actuator 8a remains positioned self-lockingly relative to the linear guide 10a, once a user's hand stops exerting a manual pushing force. The actuator 8a is accordingly displaceable in the linear guide 10a in force-fitting fashion to a limited extent. By displacing the actuator 8a, it is possible to continuously adjust an advancement of a distal portion, protruding beyond a distal tip of the capillary tube 3a, of the catheter tube 4a within the limits of the linear guide 10a. The guide element 11a can also be linearly displaceable in the linear guide 10a of the capillary housing 2a with play. Then, the catheter tube 4a is additionally secured relative to the capillary housing 2a and hence also relative to the capillary tube 3a by a clamping device, which has an elastically deformable clamping member 5a that is in the form of a sleeve and coaxially encloses the catheter tube 4a with a radial spacing. The operating member 6a provided for elastic deformation of the clamping member 5a is a clip, which is pivotably mounted on the capillary housing 2a about a pivot axis parallel to the longitudinal axis of the catheter tube 4a between a release position illustrated in FIG. 2a and a clamping position shown in FIG. 2b. In the clamping position, an inner side of the circular-arc-shaped clip 6a compresses the elastically deformable clamping member 5a in the form of a sleeve, as a result of which the catheter tube 4a is radially clamped. The operating member 6a is integrally formed on the capillary housing 2a by means of a film hinge. In the clamping position, provided for the operating member 6a is a latching mechanism, which is also integrally formed on the capillary housing 2a and manually detachably secures the operating member 6a in the clamping position on the capillary housing 2a. The actuator 8a is made large enough to enable a displacement along the capillary housing 2a using at least one finger of a user's hand.

The catheter assembly 1b according to FIGS. 3a and 3b corresponds to the catheter assembly 1a according to FIGS. 2a and 2b in terms of the functional structure. An essential difference is that, for the adjustment device, to linearly guide the actuator 8b on the capillary housing 2b, the functional track provided is a straight bar guide 10b on which the actuator 8b is guided, with limited longitudinal displacement, by means of a guide element 11b which is integrally formed on the actuator 8b and is in the form of a slider. The catheter tube 4b is mounted with linear displaceability along the capillary tube 3b of the capillary housing 2b by means of the actuator 8b, wherein a length of an advancement portion protruding distally out of the capillary tube 3b can be modified depending on the sliding position of the actuator 8b relative to the capillary housing 2b.

The catheter assembly 1b according to FIGS. 3a and 3b also has, as part of a clamping device, an elastically deformable clamping member which is in the form of a sleeve, is positioned in the region of the capillary housing 2b and coaxially encloses the catheter tube 4b. In a release position, the clamping member encloses the catheter tube 4b with a radial spacing. The clamping device has an operating member 6b which is rotatably mounted on the capillary housing 2b coaxially with a longitudinal axis of the capillary tube 3b and gives the clamping member an axial relative movability by means of a bayonet function. The clamping member is elastically deformed in the radial direction by means of a cone, which is not shown, and clamps the catheter tube 4b. Once the operating member 6b is rotated back in the opposite rotational direction again, the clamping member is re-detached from the outer surface of the catheter tube 4b.

The catheter assembly 1c according to FIG. 4 also comprises an adjustment device for adjusting the advancement of the catheter tube 4c relative to the capillary tube 3c, which is fixedly positioned relative to a capillary housing 2c. However, in the embodiment according to FIG. 4, the capillary tube 3c is fastened to a housing projection 7c, acting as an operating member of a clamping device for the catheter tube 4c, of the capillary housing 2c. The housing projection 7c is longitudinally displaceable to a limited extent relative to the capillary housing 2c in the direction of the arrow visible in FIG. 4. On the inside, the housing projection 7c accommodates the elastically deformable clamping member 5c which, upon a relative displacement coaxial with the catheter tube 4c between the housing projection 7c and the capillary housing 2c, is necessarily radially inwardly compressed owing to a conical receptacle in the housing projection 7c, as a result of which the desired clamping action on the outer surface of the catheter tube 4c is generated. When the clamping member 5c is detached, the advancement of the catheter tube 4c relative to the capillary tube 3c can be modified by means of an adjustment device. To this end, an annular actuator 8c longitudinally displaceably mounted in a linear guide 10c of the capillary housing 2c is fastened on the outer surface of the catheter tube 4c. An actuating element 12c, which is in the form of a slide, is form-fittingly connected to the actuator 8c and is also guided in the linear guide 10c of the capillary housing 2c parallel to the longitudinal axis of the catheter tube 4c with limited displaceability, engages on the actuator 8c. The actuating element 12c has a guide element 11c in the form of a bar, this guide element on the one hand form-fittingly blocking a distal end face of the actuator 8c and on the other hand being longitudinally displaceably guided in the linear guide 10c of the capillary housing 2c. Additionally provided are two elastically flexible fingers, not denoted in more detail, which are integrally formed on the actuating element 12c and brace the opposite end face of the actuator 8c against the guide bar 11c. The two fingers are inclined such that the catheter tube 4c can be pushed into the capillary housing 2c from a proximal end of the capillary housing 2c and then into the capillary tube 3c distally through the housing projection 7c. The two fingers deflect and latch behind the actuator 8c again, with the result that the catheter tube 4c is secured relative to the actuating element 12c.

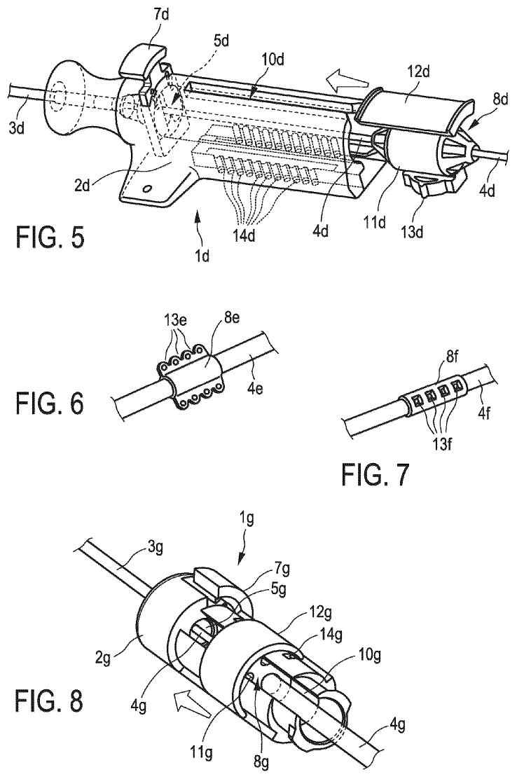

In the case of the catheter assembly 1d according to FIG. 5, a linear guide 10d is provided in a capillary housing 2d and extends in the manner of a tube coaxially with a longitudinal axis of the capillary tube 3d fastened distally to the capillary housing 2d. The catheter tube 4d is enclosed by an actuator 8d, which additionally forms a partially cylindrical guide element 11d longitudinally displaceably guided in the linear guide 10d of the capillary housing 2d. Additionally, as actuating element 12d, integrally formed on the actuator 8d is a slide, which is guided in a longitudinal slot of the linear guide 10d of the capillary housing 2d and protrudes beyond a top side of the capillary housing 2d. On an opposite, bottom side, the actuator 8d is provided on either side with an integrally formed-on, elastically flexible latching bar 13d, which is associated with complementary latching cutouts 14d inside the capillary housing 2d. This ensures a number of latching stages for an advancement of the catheter tube 4d relative to the capillary tube 3d that corresponds to the number of the latching cutouts 14d. The latching bars 13d engaging in the respective latching cutout 14d on opposite sides of the actuator 8d secure the respective adjusted advancement position. Moreover, as and when required, the catheter tube 4d is axially fixed by the clamping device, which has an operating member 7d and a radially elastically deformable clamping member 5d. The operating member 7d is, in the manner of a push pin, guided in the capillary housing 2d orthogonally to the longitudinal axis of the catheter tube 4d with the ability to perform a limited stroke movement. The operating member 7d radially acts on the clamping member 5d, which causes the latter to be radially compressed and pressed against the outer surface of the catheter tube 4d. The operating member 7d is assigned, in a way not illustrated in more detail, a latching position for the clamping position in the capillary housing 2d, in which position the clamping member 5d securely clamps the catheter tube 4d.

In the embodiments according to FIGS. 6 and 7, actuators 8e and 8f are fastened on the respective catheter tube 4e and 4f and are each provided with multiple latching profiles 13e and 13f that extend in the longitudinal direction of the catheter tube 4e, 4f. In a way not illustrated in more detail, to that end the capillary housing has complementary movable latching elements, as a result of which the actuator 8e, 8f can be manually detachably fixed relative to the capillary housing in different latching positions in the advancement direction of the catheter tube 4e, 4f.

The catheter assembly 1g according to FIG. 8 largely corresponds in terms of function to the catheter assembly 1d according to FIG. 5. It is also the case here that an annular actuator 8g is fastened on an outer surface of the catheter tube 4g and is longitudinally displaceable in the capillary housing 2g and the capillary tube 3g fastened thereto. In the case of the catheter assembly 1g, the actuator 8g is fixed where the catheter tube 4g merges into an extension hose, which is fastened proximally with respect to a distal tube portion of the catheter tube 4g. The actuator 8g is longitudinally displaceably guided by way of an annular guide face in a cylindrical linear guide 10g of the capillary housing 2g. The actuator element provided is a slide 12g, which is integrally connected to the actuator 8g by means of a guide bar 11g. The guide member 11g protrudes upwards through a longitudinal slot of the linear guide 10g of the capillary housing 2g. The slide 12g is longitudinally displaceably guided on the outside of a cylindrical housing portion of the capillary housing 2g. Provided on the cylindrical housing portion of the capillary housing 2g are multiple latching recesses 14g arranged one behind another at regular intervals in the longitudinal direction. The slide 12g has at least one complementary latching lug, not illustrated in more detail, which manually detachably latches into the respective latching recess 14g of the capillary housing 2g depending on an advancement position of the catheter tube 4g relative to the capillary tube 3g. In addition, provided distally in the capillary housing 2g is a clamping member 5g, which is in the form of a sleeve and, analogously to the above-described clamping members, is elastically deformable and coaxially encloses the catheter tube 4g. Similarly to the embodiment according to FIG. 5, an operating member 7g is mounted on the capillary housing 2g with the ability to perform a radial stroke movement, in order to be pressed radially inwards, resulting in the radial deformation of the clamping member 5g, for a clamping function. This causes the catheter tube 4g to be radially clamped, and secured relative to the capillary housing 2g.

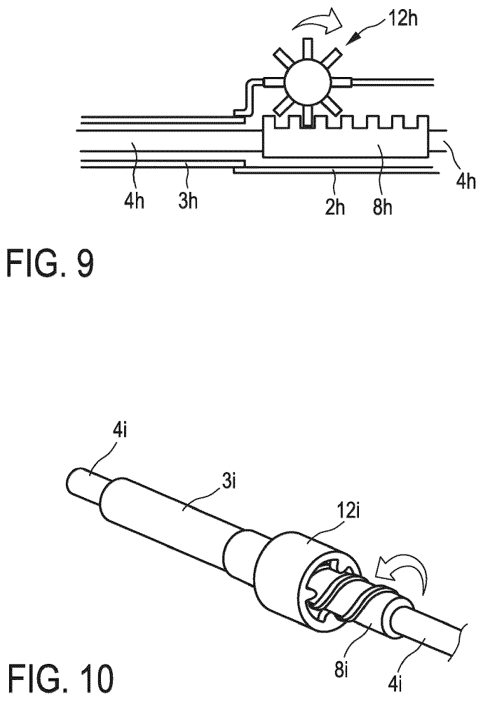

In the embodiment according to FIG. 9, an actuating element 12h of an adjustment device for modifying the advancement of the catheter tube 4h is rotatably mounted on the capillary housing 2h about an axis of rotation orthogonal to the advancement direction of the catheter tube 4h. On a longitudinal side facing towards the actuating element 12h, the actuating member 8h has a toothed-rack function. The actuating element 12h is provided with a complementary face toothing which meshes with the toothed-rack function of the actuator 8h, in order to obtain the desired advancement function for the catheter tube 4h in the capillary tube 3h.

In the embodiment according to FIG. 10, an advancing movement of the catheter tube 4i relative to a capillary tube 3i is obtained by an actuating element 12i which is rotatably mounted on a capillary housing, not illustrated in more detail, coaxially with the capillary tube 3i. The actuator 8i has on its outer circumference a threaded worm, which interacts with an internal thread of the actuating element 12i. A relative rotation between the actuating element 12i and the actuator 8i thus necessarily leads to a continuous advancement movement in the distal direction or in the proximal direction, depending on the relative rotation between the actuating element 12i and the actuator 8i.

In the case of a catheter assembly according to FIG. 11, a capillary tube 3 in which a catheter tube 4 is longitudinally displaceably guided is fastened distally to a capillary housing 2. Distally from the capillary housing, fastened on an outer surface of the catheter tube 4 is an actuator 8, which is in the form of a bead and forms a radial projection relative to a longitudinal axis of the catheter tube 4. In a manner not illustrated in more detail, provided in the capillary housing 2 is an elastically deformable clamping member, which can be twisted by means of a rotatable actuating wing 7, in order to have the effect of elastically clamping the catheter tube 4. A user can thus use one hand, with the clamping device in the detached state, to act on the actuator 8 and longitudinally displace the catheter tube 4 relative to the capillary tube 3. The correspondingly adjusted advancement can be fixed by subsequently rotating the actuating wing 7 using the same hand because, owing to the rotation of the actuating wing 7, the clamping member applies a clamping force radially from the outside to the outer surface of the catheter tube 4.

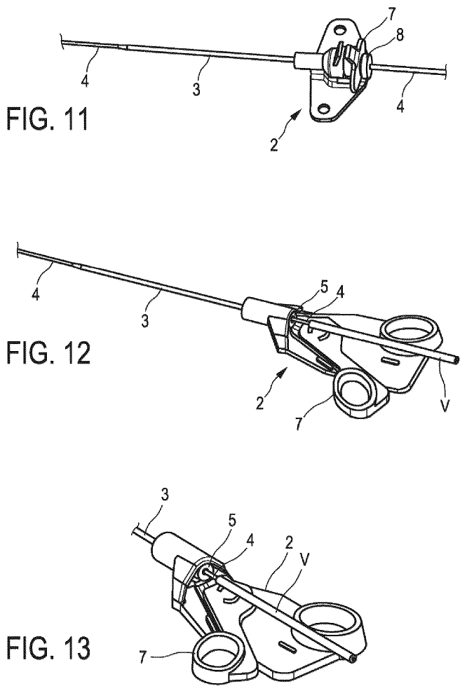

In the case of the catheter assembly according to FIGS. 12 and 13, an extension hose V of larger diameter is pushed onto a proximal end of a catheter tube 4 and force-fittingly fixed on the proximal face end of the catheter tube 4. Since the extension hose V has a larger diameter than the catheter tube 4, necessarily provided by the pushed-on distal face end of the extension hose V is a radial projection on the catheter tube 4, this radial projection being necessarily itself guided with limited displaceability relative to the capillary housing 2 upon a displacement of the catheter tube. The capillary housing 2 has a capillary tube 3 attached distally to the capillary housing 2. Provided coaxially with the catheter tube 4, on the capillary housing 2, is a radially elastically deformable clamping member 5, which is in the form of a sleeve and distally forms an axial stop for the radial projection of the extension hose V. The clamping member 5, as part of a clamping device, is elastically deformed by a scissors wing, which serves as an actuating element 7 and is pivotable relative to the capillary housing 2. The capillary housing 2 has, as counter wing to the scissors function of the scissors wing, an integrally formed engagement point, in which a finger of a user's hand can engage. Using two fingers of one hand, it is thus possible to pivot the scissors wing relative to the counter wing, in order to elastically deform the clamping member and thus axially fix the catheter tube 4 relative to the capillary housing 2. Using the same hand, the extension hose V can be gripped before the clamping member 5 is clamped and an advancement of the catheter tube 4 relative to the capillary tube 3 can be adjusted. Corresponding latching points on the actuating wing 7, on one hand, and on the capillary housing 2, on the other hand, can ensure the elastic deformation of the clamping member 5 and thus the clamping position of the scissors wing when the scissors parts are pressed together, with the result that the catheter tube 4 remains axially fixed.

1. A catheter assembly for regional anaesthesia, the catheter assembly comprising:
a capillary housing on which distally a capillary tube is provided;
a catheter tube inserted proximally in the capillary tube and protruding distally partially out of the capillary tube;
a clamping device that is manually actuatable for detachable fixing of the catheter tube relative to the capillary tube and the capillary housing;
an adjustment device on the capillary housing, the adjustment device ensuring a graduated or continuous manual displacement of the catheter tube in the capillary tube; and
at least one radial projection that is manually grippable on the catheter tube, the at least one radial projection being guided with limited displaceability along the capillary housing,
the catheter tube being arranged in the capillary tube with limited linear displaceability.

2. The catheter assembly according to claim 1 further comprising an actuating element that is manually operable and operatively connected to the at least one radial projection, the actuating element being movably arranged on the capillary housing for a displacement of the at least one radial projection along the capillary housing.

3. The catheter assembly according to claim 2, wherein the at least one radial projection is an actuator.

4. The catheter assembly according to claim 3, wherein the actuating element is rotatably mounted on the capillary housing and operable to displace the actuator and the catheter tube along the capillary housing and in the capillary tube.

5. The catheter assembly according to claim 4, wherein the actuating element is:
rotatably mounted on the capillary housing coaxially with a longitudinal axis of the capillary tube, and
coupled to the actuator by a screw drive.

6. The catheter assembly according to claim 5, wherein:
the actuating element is rotatably mounted on the capillary housing about an axis of rotation orthogonal to the longitudinal axis of the capillary tube, and
the actuating element is coupled to the actuator by a toothed-rack drive.

7. The catheter assembly according to claim 3, wherein the actuating element is fixedly connected to the actuator.

8. The catheter assembly according to claim 3, wherein the actuating element interacts with the actuator with transmission of movement.

9. The catheter assembly according to claim 2, wherein the actuating element is displaceably mounted on the capillary housing.

10. The catheter assembly according to claim 2, wherein the actuating element is manually detachably retained on the capillary housing in force-fitting or form-fitting fashion in at least two different functional positions.

11. The catheter assembly according to claim 10, further comprising mutually complementary latching profiles on the actuating element and on the capillary housing, the mutually complementary latching profiles configured to manually detachably secure the at least two different functional positions.

12. The catheter assembly according to claim 2, further comprising a linear guide on the capillary housing for displacement of the actuating element, wherein the actuating element is an actuating slide that is displaceable along the linear guide.

13. The catheter assembly according to claim 1, wherein the clamping device has a clamping member that force-fittingly acts on the catheter tube.

14. The catheter assembly according to claim 13, further comprising an operating member that is movably mounted on the capillary housing, the operating member configured to elastically deform the clamping member into a clamping position, in order to force-fittingly clamp the catheter tube, and into a release position, in order to release the clamping member such that the clamping member returns to an unloaded initial position in which the catheter tube is movable relative to the clamping member.

15. The catheter assembly according to claim 14, wherein the operating member is linearly movably, pivotably or rotatably mounted on the capillary housing.
